(19) United States
(12) Patent Application Publication
(10) Pub. No.: US 2003/0088181 A1
(43) Pub. Date: May 8, 2003

(54) Method of localizing an object in an MR apparatus, a catheter and an MR apparatus for carrying out the method

(76) Inventor: Bernhard Gleich, Hamburg (DE)

Correspondence Address: THOMAS M. LUNDIN, Philips Medical Systems (Cleveland), Inc., 595 Miner Road, Cleveland, OH, 44143, US

(21) Appl. No.: 10/274,224

(22) Filed: Oct. 18, 2002

(30) Foreign Application Priority Data
Oct. 19, 2001 (DE) 10151779.3

(51) Int. Cl. A61B 6/00; A61B 5/05; A61M 25/00

(52) U.S. Cl. 600/434; 600/411; 600/415

(57) ABSTRACT

A method for localizing an object, preferably a medical instrument and notably a catheter (60) introduced into a body in the examination volume of an apparatus operating on the basis of magnetic resonance (MR), evaluates the interaction between an electromagnetic resonant circuit (6), mounted on the object (60), and an RF field applied in the MR apparatus for nuclear magnetization of the body. In such a method a simple, fast and accurate localization of an object, notably a catheter, in an image-forming MR device is enabled in that use is made of a resonant circuit (6) which is tuned to the frequency of the RF field and is capable of assuming two states with a different resonant circuit quality factor, in that in a first state nuclear magnetization with a flip angle is produced by means of a first RF pulse while the resonant circuit (6) is in one of the two states, and that in a second state a second RF pulse is applied so as to rephase the nuclear magnetization while the resonant circuit (6) is in the other one of the two states.

### BACKGROUND

The present invention relates to the field of magnetic resonance tomography. It concerns a method of localizing an object, in particular a catheter, which is present in a body in the examination zone of an apparatus operating on the basis of magnetic resonance (MR). The invention also relates to a catheter and to an MR apparatus for carrying out the method.

A method and devices of the kind set forth are known, for example, from EP A2 0 928 972.

Image-forming magnetic resonance methods (Magnetic Resonance Imaging or MRI) which utilize the interaction between magnetic fields and nuclear spins in order to form two-dimensional or three-dimensional images are widely used nowadays, notably in the field of medical diagnostics, because for the imaging of soft structures they are superior to other imaging methods in many respects, do not require ionizing radiation and usually are not invasive.

According to the MR method, the body to be examined is arranged in a strong, uniform magnetic field whose direction at the same time defines an axis (normally the z axis) of the co-ordinate system on which the measurement is based. The magnetic field produces different energy levels for the individual nuclear spins in dependence on the magnetic field strength which can be excited (spin resonance) by application of an electromagnetic alternating field of defined frequency (Larmor frequency). From a macroscopic point of view the distribution of the individual nuclear spins produces an overall magnetization which can be deflected out of the state of equilibrium along a spiral-shaped path by application of an electromagnetic pulse of appropriate frequency (RF pulse) while the magnetic field extends perpendicularly to the z axis, so that it performs a precessional motion about the z axis. The precessional motion describes a surface of cone whose angle of aperture is referred to as the flip angle. The magnitude of the flip angle is dependent on the strength and the duration of the applied electromagnetic pulse. In the case of a so-called 90° pulse, the spins are deflected from the z axis to the transverse plane (flip angle 90°).

After termination of the RF pulse, the magnetization relaxes back again to the original state of equilibrium, in which the magnetization in the z direction is built up again with a first time constant T₁ (spin lattice relaxation time) and the magnetization in the direction perpendicular to the z direction relaxes with a second time constant T₂ (spin-spin relaxation time). The variation of the magnetization can be detected by means of a coil which is customarily oriented in such a manner that the variation of the magnetization is measured in the direction perpendicular to the z axis (transverse magnetization, time constant T₂). The decay of the transverse magnetization is accompanied, after application of a 90° pulse, by a transition of the nuclear spins (induced by local magnetic field inhomogeneities) from an ordered state with the same phase to a state of equilibrium in which all phase angles are uniformly distributed (dephasing). The dephasing can be compensated by means of a refocusing pulse (180°) pulse. This produces an echo signal (spin echo) in the detection coil.

In order to realize spatial resolution in the body, linear gradient fields extending along the three main axes are superposed on the uniform magnetic field, leading to a linear spatial dependency of the spin resonance frequency. The signal picked up in the detection coil then contains components of different frequencies which can be associated with different locations in the body after Fourier transformation from the time axis to the frequency axis.

An imaging MR method of this kind can at the same time be used to localize or track the motion of a medical instrument introduced into the body, notably a catheter.

Therefore, it has been proposed (see EP A2 0 928 972) to provide a closed resonant circuit which consists of a microcoil and a capacitor on the tip of the instrument in order to localize the instrument (catheter). The resonant circuit, being tuned to the frequencies occurring during the MR method, locally increases the RF signal, and hence the flip angle in the body, because of its resonance properties. This local increase can be detected during MR imaging so as to be used for localizing the tip of the instrument. However, in this method a problem is encountered in that the localization of the tip of the instrument forms part of the imaging process and hence takes place comparatively slowly. Moreover, the superposition of the RF signal amplified by the resonant circuit and the RF signals occurring in the remainder of the body impedes the detection and localization.

Another solution for localizing (DE A1 199 56 595) utilizes a non-linear resonant circuit with a microcoil. The excitation of the nuclear magnetization is carried out by means of an RF pulse whose frequency spectrum does not overlap the range of the spin resonance spectrum, so that no magnetic resonance is excited outside the near field of the microcoil. Because of its non-linearity, however, the non-linear resonant circuit generates an RF signal from the RF pulse, which RF signal locally overlaps the spin resonance frequency and causes local excitation of the magnetic resonance. This local nuclear magnetization can be measured and the position determined can be reproduced in the MR image of the body being examined.

This method has the drawback that it is necessary to operate with a frequency other than the resonance frequency (Larmor Frequency) and notably that the generating of the Larmor frequency in the form of higher harmonics by the non-linear resonant circuit is not very effective and yields low field strengths only.

### SUMMARY

Therefore, it is an object of the invention to provide a method, a catheter and an MR apparatus which enable simple, fast and accurate localization in a body to be examined.

In accordance with one aspect of the invention, the object is achieved by a method of localizing an object, preferably a medical instrument and in particular a catheter which is present in a body in the examination zone of an apparatus operating on the basis of magnetic resonance (MR), which method evaluates the interaction between an electromagnetic resonant circuit, which is provided on the object and does not include supply leads, and an RF field which is applied for nuclear magnetization in the body in the MR apparatus, and use is made of a resonant circuit which is tuned to the frequency of the RF field and is capable of assuming two states with a different resonant circuit quality factor, that in a first state a nuclear magnetization with a flip angle is induced by means of a first RF pulse while the resonant circuit is in one of the two states, and that in a second step the nuclear magnetization is rephased by means of a second RF pulse while the resonant circuit is in the other one of the two states.

In accordance with a more limited aspect of the invention, the object is achieved by a catheter, which catheter includes a resonant circuit without supply leads which is provided with an inductance, notably in the form of a microcoil, and a capacitance which is connected parallel to the inductance, and an additional switching element is provided in the resonant circuit, which switching element influences the resonant circuit quality factor of the resonant circuit.

In accordance with another aspect of the invention, the object is achieved by an MR apparatus, which apparatus includes first means for generating a uniform, steady magnetic field whose strength defines the Larmor frequency, second means for generating RF pulses, and third means for receiving MR signals generated in the object to be examined, and a control unit for controlling the RF pulses in such a manner that there is generated a temporal succession of a first RF pulse, having a first amplitude and a first pulse duration, for nuclear magnetization with a flip angle and a second RF pulse, having a second amplitude and a second pulse duration, for rephasing the nuclear magnetization.

The basic idea of the invention is to provide the catheter to be localized with a resonant circuit without supply leads, which resonant circuit may have different resonance quality factors. When the nuclear magnetization is excited by a first RF pulse and is rephased again by a second, subsequent RF pulse and the resonant circuit has a different resonance quality factor for the first and the second RF pulse, no net effect prevails outside the coil associated with the resonant circuit; however, a net magnetization remains in the near field of the coil because of the different resonance step-up of the field, which net magnetization can be evaluated for the localization in the MR equipment. This type of differential measurement enables fast and exact real-time localization which does not necessitate the formation of a complete image, is safe for the patient, can be readily carried out and does not require additional equipment. Moreover, because of the speed, the localization is not subject to motional effects to a high degree.

In conformity with a preferred version of the method in accordance with the invention, the resonant circuit can be switched between the two states and is switched between the two states in the course of the localization. This enables unambiguous differentiation which can be suitably evaluated.

The resonant circuit is preferably switched over by an RF pulse, switching-over notably utilizing one of the two RF pulses required for the nuclear magnetization or the rephasing thereof. This offers the advantage that no additional devices are required so as to influence the resonant circuit.

A preferred further version of this method is characterized in that two RF pulses are used for the nuclear magnetization and the rephasing thereof, one of said RF pulses having a low power and a long pulse duration while the other has a high power and a short pulse duration, and that the RF pulse having the high power and the short pulse duration is used to switch over the resonant circuit.

A very simple implementation of the invention is obtained when a non-linear element which is included in the resonant circuit is used for switching over.

However, it is also feasible to provide the resonant circuit with an optically controllable element and to switch over the resonant circuit by means of an optical signal.

In a preferred embodiment of the catheter in accordance with the invention the switching element is formed by a diode, that is, either a simple diode which is connected in parallel with the capacitor, or a capacity diode which is connected in series with the capacitance, or a photodiode; in that case a series connection of the photodiode and a further capacitance is connected in parallel with the capacitance.

### DRAWINGS

Embodiments of the invention will be described in detail hereinafter, by way of example, with reference to the drawing. Therein:

FIG. 1 is a strongly simplified representation of an MR apparatus which is suitable for carrying out the method in accordance with the invention;

FIG. 2 shows the circuit diagram of such an apparatus;

FIG. 3 shows the circuit diagram and the arrangement in a catheter of a resonant circuit in accordance with a first embodiment of the invention;

FIG. 4 shows an exemplary sequence of RF pulses for localization in conformity with the method in accordance with the invention, and

FIG. 5 shows the circuit diagram of a second embodiment of a resonant circuit in accordance with the invention.

### DESCRIPTION

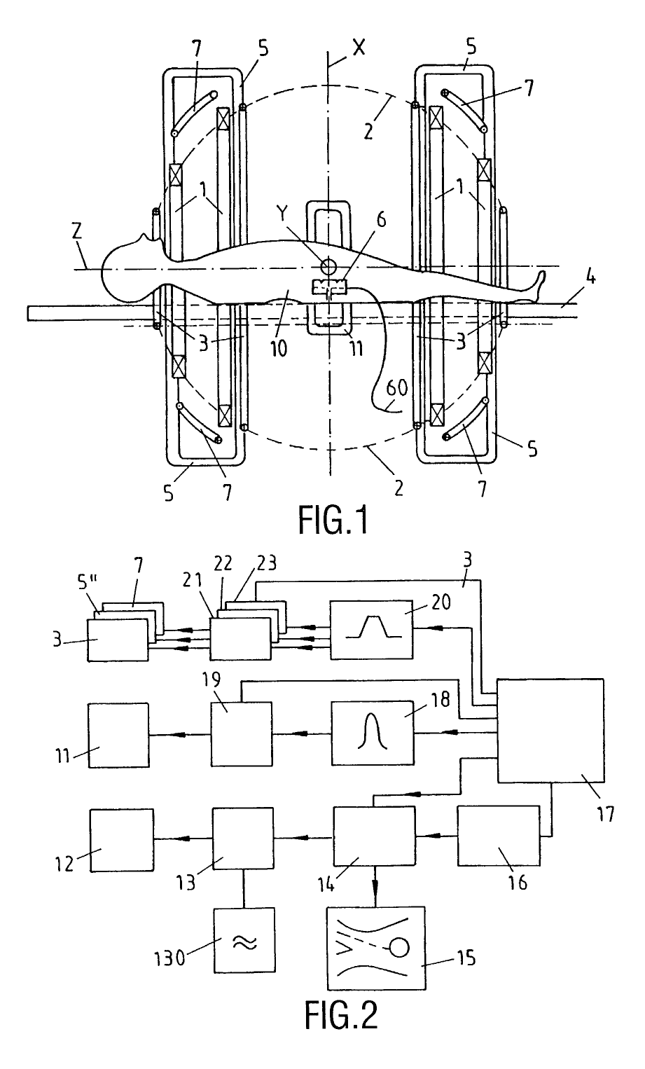

The FIGS. 1 and 2 show an MR apparatus which is suitable for carrying out the method in accordance with the invention. The device for forming MR images as shown in FIG. 1, also referred to as a magnetic resonance examination apparatus, includes a system which consists of four main coils 1 and serves to generate a uniform, steady magnetic field in the z direction (main field) whose magnetic flux density (magnetic induction) may be of the order of magnitude of from some tenths of Tesla to some Tesla. The main coils 1, being arranged so as to be concentric with the z axis, may be situated on a spherical surface 2. An object to be examined, for example, a patient 10 who is positioned on a table top 4, is arranged within these coils.

Four first coils 3 are arranged on the spherical surface 2 or on a cylindrical surface in order to generate a first gradient magnetic field which extends in the direction of the z axis and varies linearly in this direction. Furthermore, four second coils 7 are provided which generate a second gradient magnetic field which also extends in the direction of the z axis but varies linearly in the vertical direction (x direction). Finally, using four third coils 5 (only two of which are shown) there is generated a third gradient magnetic field which extends in the direction of the z axis and varies linearly in the plane perpendicular to the plane of drawing of FIG. 1 (y direction).

A medical instrument 60 (for example, a catheter) is introduced into the part of the patient to be examined; at the tip of said instrument there is provided a resonant circuit 6. This part of the body is also enclosed by an RF transmission coil 11 whereto an RF pulse can be applied and whereby this part is traversed by an RF magnetic field which excites spin resonance. The relaxation subsequent to said excitation causes a change of the magnetization states which induces a corresponding voltage in an RF receiving coil 12 (see FIG. 2); this voltage is evaluated for the purpose of MR imaging and the gradient magnetic fields enable localization of the excited states.

The components for the operation of the described device are diagrammatically represented in FIG. 2 and include a control unit 17, controlling a gradient waveform generator 20; to the outputs of this generator there are connected a first, a second and a third gradient amplifier 21, 22, 23, respectively. These amplifiers generate the respective currents for the first, the second and the third coil 3, 5, 7, respectively. The gain factors of these amplifiers can be adjusted independently of one another by the control unit 17, via leads 39, so that the coils 3, 5, 7 generate the gradient fields in the x, y and z directions and slice selection can be performed in the corresponding three spatial directions in the zone being examined.

Furthermore, the control unit 17 controls an RF generator 18 in order to adjust the frequency of the RF pulses to the Larmor frequencies which are dependent on the gradient fields and to generate RF pulses of different length for the MR imaging. The RF pulses are applied to an amplifier 19, whose gain is controlled by the control unit 17, and subsequently reach the RF transmission coil 11.

The MR signals induced in the RF receiving coil 12 by the relaxation of the excited magnetization states are demodulated in a quadrature demodulator 13 by mixing with two 90° mutually offset carrier oscillations (with a Larmor or MR frequency determined by the local strength of the steady magnetic fields) from an oscillator 130, thus producing two signals which may be considered to be the real component and the imaginary component of a complex signal. These signals are applied to an analog-to-digital converter 14. Finally, an image processing unit 16 reconstructs the MR images in known manner for display on a monitor 15.

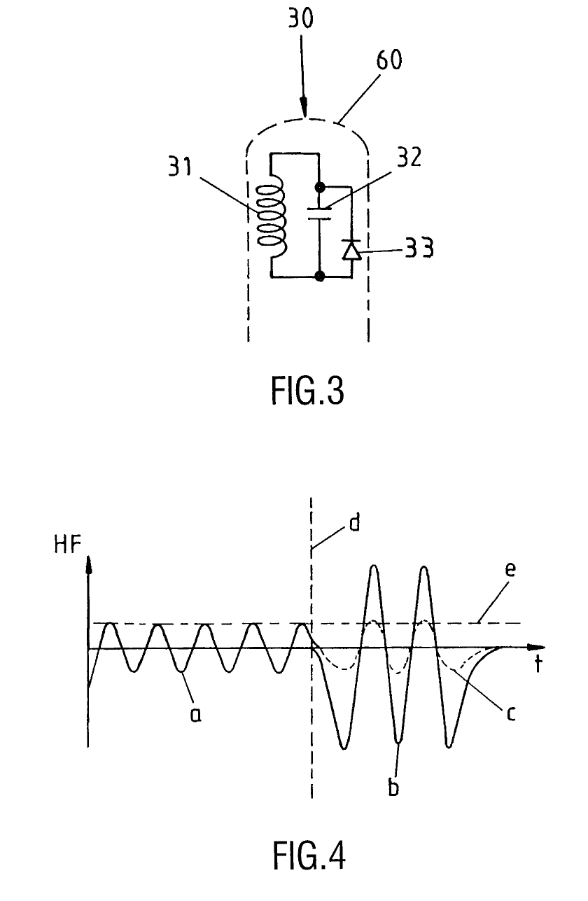

The resonant circuit 6 of FIG. 1, not requiring any electrical leads to the environment, may have various forms; FIG. 3 shows the circuit diagram of a resonant circuit 30 which is provided in the tip of a catheter 60 (represented by a dashed line). The catheter 60 is introduced into the body of a person (patient) 10 to be examined who is arranged in the examination zone of the MR apparatus shown in FIG. 1. The resonant circuit 30 includes an inductance 31, preferably being a microcoil, and a capacitance 32 which is connected in parallel therewith. The inductance 31 and the capacitance 32 form a parallel resonant circuit which is tuned essentially to the Larmor frequency of the body material excited by the MR apparatus. A non-linear element in the form of a diode 33 is connected parallel to the capacitance 32.

The resonant circuit 30 is subject to an RF pulse transmitted by the MR apparatus for the excitation of the nuclear magnetization. When the RF power of the pulse is low, the voltage across the diode 33 is small. In that case the diode 33 is not conductive, because its threshold voltage is not exceeded. The resonance quality factor of the resonant circuit 30 is then comparatively high and the local RF field at the area of the microcoil 31 is then multiplied by a factor G₁>>1. When the RF power is significantly increased, the diode 33 becomes conductive and reduces the resonance quality factor by way of the associated bypass function: the local RF field is not increased to such a high degree; the multiplication factor then assumes a value G₂<G₁.

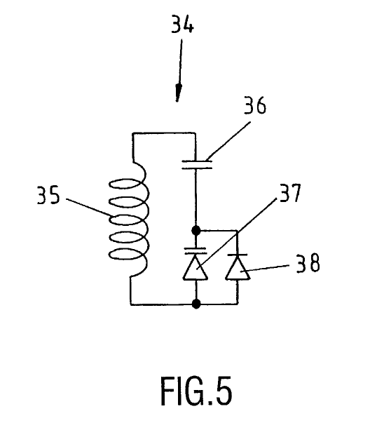

This behavior of the resonant circuit 30 can be used to realize a differential method; the behavior of the resonant circuit 30 is diagrammatically shown in FIG. 4 on the basis of the variation of the local RF field (RF) in time (t). When in a first step a long RF pulse having a comparatively low RF power is used to rephase the magnetization at the area of the catheter 60, the flip angle at the tip of the catheter 60, that is, at the area of the microcoil 31, is substantially increased by the resonance step-up (factor G₁; curve a in FIG. 4; no clipping by the diode commences at the curve e). When subsequently (as from the boundary line d in FIG. 4) a second brief RF pulse (curve b in FIG. 4) with a 180° shifted phase and a comparatively high RF power is applied so as to rephase the magnetization, the excited magnetization in the area outside the microcoil 31 becomes zero when the time integrals of the RF pulses are the same. In the direct vicinity of the microcoil 31, however, the magnetization is not equal to zero because the effect of the resonance step-up is smaller due to the clipping behavior of the diode 33 (factor G₂; the RF field defined by the diode extends along the curve c in FIG. 4).

The tip of the catheter 60 appears as a single peak in a projection. In this context a projection is to be understood to mean that the RF pulse excites a volume (in the examination zone). At the instant at which the echo (spin echo) occurs, a magnetic field gradient is applied in a projection direction. Fourier transformation of the signal obtained yields a projection, that is, the signal intensity distribution which results from the integration of the slices perpendicular to the projection direction is plotted along the space co-ordinate. When a distinct peak, caused by the catheter 60, can be detected in such a projection, its position in one spatial direction is found by this measurement. The position in space of the catheter 60 is determined by carrying out a total of three measurements in three orthogonal spatial directions. This localization by projection is very fast. However, if the localization of the catheter 60 were performed by acquisition of a complete image, 256 or more of these steps would be required (for the formation of a complete image) and even then it would not be certain that the catheter would indeed be detected.

During the localization it is also possible to select a given slice of the examination zone by application of a magnetic field gradient, thus enabling individual instruments with their respective resonant circuits (markers) to be distinguished when a number of marked instruments are present. Before the second RF pulse is applied so as to rephase the magnetization, however, refocusing of the spins must then be carried out in known manner by means of a refocusing pulse.

If no specific volume of the examination zone is selected, the measuring sequence includes the following steps:

RF pulse with power 1;

RF pulse with power 2 which rephases the magnetization remote from the catheter;

possibly refocusing pulses in the case of spin echo;

gradient in projection direction;

data acquisition.

However, when a given volume of the examination zone is selected, the following measuring sequence is obtained:

gradient localization sequence with RF pulse with power 1 (with or without refocusing, depending on the progression);

RF pulse with power 2 which rephases the magnetization remote from the catheter; a gradient sequence is then applied which selects the same volume;

possibly a refocusing pulse in the case of spin echo;

gradient in the projection direction;

data acquisition.

It is also possible to use other non-linear components in the resonant circuit 30 instead of the diode 33; feasible components in this respect are semiconductor components with a plurality of PN junctions such as, for example, transistors, field effect components (FETs in which the source and gate are interconnected), or so-called Wollaston wires, that is, extremely thin wires of platinum or the like which are extremely quickly heated when exposed to current, thus increasing their resistance. Also feasible are various thermal resistors (PTC, NTC), provided they are constructed so as to be small enough, and capacitors with saturable dielectrica (ferroelectrica). However, diodes are most suitable by far.

If the image formed by the MR apparatus is not to be disturbed by the markers, use may be made of an alternative resonant circuit which is switched over to the state with a large resonance step-up by a brief RF pulse of high power. FIG. 5 shows an embodiment of such a resonant circuit. The resonant circuit 34 as shown in FIG. 5 again includes a microcoil 35 and a capacitance 36 in a parallel connection. Within the parallel connection there is provided a capacitance diode (varicap diode) 37 which is connected in series with the capacitance, a further diode 38 being connected parallel to said capacitance diode. The diode 38 should have an as small as possible forward voltage. In the rest state the capacitance diode 37 has a high capacitance and the resonance frequency of the resonant circuit 34 is low. The capacitance diode 37 is charged by a strong RF pulse (in this case also a pulse which has a low frequency) and reduces its capacitance. The resonance frequency of the resonant circuit 34 then increases. This state prevails for a brief period of time.

The described differentiation method, in which first a first RF pulse rotates the magnetization which is subsequently rephased again by a second RF pulse, is suitable for all markers which can be switched between the pulses in order to influence the magnetization. An optically switchable resonant circuit forms an example of a further switchable marker. A resonant circuit of this kind has a construction which is similar to that shown in FIG. 3, be it that the diode 33 is replaced by a photodiode. Via a light conductor an optical pulse is then applied to the catheter 60 between the RF pulses, which optical pulse makes the photodiode conductive and hence reduces the quality factor of the resonant circuit.

Generally speaking, the advantage of the differential method consists in that motion-imposed effects on the MR image are eliminated to a high degree, because the differentiation takes place within one millisecond.

Overall, the invention offers the following advantages:

the localization is exact;

the localization can be carried out in real time;

the method is RF safe for a patient;

the markers can be readily constructed and no additional devices are required in the MR apparatus;

no motion-imposed disturbances occur in the difference image.

The invention has been described with reference to the preferred embodiment. Obviously, modifications and alterations will occur to others upon reading and understanding the preceding description. It is intended that the invention be construed as including all such modifications and alterations insofar as they come within the scope of the appended claims or the equivalents thereof.

What is claimed is:

1. A method of localizing a medical instrument in a body in an examination zone of a magnetic resonance apparatus, the method comprising the steps of:
evaluating the interaction between an electromagnetic resonant circuit disposed on the medical instrument and an RF field which is applied for nuclear magnetization in the body in the examination zone; and
tuning the resonant circuit to the frequency of the RF field, the resonant circuit being capable of assuming two states with a different resonant circuit quality factor;
inducing a first state of nuclear magnetization with a flip angle by means of a first RF pulse while the resonant circuit is in one of the two states; and
rephasing the nuclear magnetization by means of a second RF pulse while the resonant circuit is in the other one of the two states.

2. A method as claimed in claim 1, wherein the resonant circuit can be switched between the two states, and the resonant circuit is switched between the two states in the course of the localization.

3. A method as claimed in claim 2, wherein the resonant circuit is switched by an RF pulse, and for the switching use is made of one of the two RF pulses required for the nuclear magnetization or for the rephasing thereof.

4. A method as claimed in claim 3, wherein two RF pulses are used for the nuclear magnetization and for the rephasing thereof, one of said RF pulses having a low power and a long pulse duration while the other RF pulse has a high power and a short pulse duration, and the RF pulse having the high power and the short pulse duration is used for switching the resonant circuit.

5. A method as claimed in claim 4, wherein a non-linear element is included in the resonant circuit and is used for the switching.

6. A method as claimed in claim 4, wherein the first RF pulse applied is the RF pulse having the low power and the long pulse duration, the second RF pulse applied is the RF pulse having the high power and the short pulse duration, and the second RF pulse switches the resonant circuit from a state with a high resonant circuit quality factor to a state with a low resonant circuit quality factor.

7. A method as claimed in claim 2, wherein the resonant circuit includes an optically controllable element and is switched over by an optical signal.

8. A catheter for use with a magnetic resonance imaging apparatus, the catheter comprising:
a resonant circuit having an inductance in the form of a microcoil;
a capacitance connected parallel to the inductance; and
a switching element provided in the resonant circuit, the switching element for influencing the resonant circuit quality factor of the resonant circuit.

9. A catheter as claimed in claim 8, wherein the switching element is a diode.

10. A catheter as claimed in claim 8 wherein the resonant circuit does not have supply leads.

11. A magnetic resonance apparatus comprising:
a main magnet for generating a substantially uniform, steady magnetic field in an examination region;
a gradient coil system for generating gradient fields in the examination region;
an RF transmit coil for transmitting at least first and second RF signals into the examination region and exciting spin resonance in an object disposed within the examination region;
an RF receive coil for receiving RF signals, induced by the RF transmit coil, from the examination region; and
a medical device, the medical device comprising:
a resonant circuit disposed on the medical device, the resonance circuit including an inductance, a capacitance connected in parallel with the inductance, and a non-linear element in electrical connection with the inductance and capacitance.

12. A magnetic resonance apparatus according to claim 11 wherein:
the inductance comprises a microcoil;
the capacitance comprises a capacitor; and
the non-linear element comprises a diode.

13. A magnetic resonance apparatus according to claim 11 wherein the medical device does not have electrical supply lines.

14. A magnetic resonance apparatus according to claim 11 wherein the first RF signal comprises a first power and a first duration and the second RF signal comprises a second power and a second duration, the second power being greater than the first power and the second duration being less than the first duration.

15. A magnetic resonance apparatus according to claim 14 wherein the non-linear element is not conductive in response to the first RF signal and is conductive in response to the second RF signal.

16. A magnetic resonance apparatus according to claim 14 wherein the resonant circuit has a first quality factor in response to the first RF signal and a second quality factor in response to the second RF signal, the first quality factor being greater than the second quality factor.

17. A magnetic resonance apparatus according to claim 11 wherein the medical device further comprises a capacitance diode electrically connected in series with the capacitance and electrically connected in parallel with the non-linear element.
